(19) United States
(12) Patent Application Publication
(10) Pub. No.: US 2025/0137989 A1
(43) Pub. Date: May 1, 2025

(54) METHOD FOR EVALUATING COMPREHENSIVE PERFORMANCE OF ACOUSTO-OPTIC CHALCOGENIDE GLASS MATERIAL

(71) Applicant: Ningbo University, Ningbo City (CN)

(72) Inventors: Shixun DAI, Ningbo City (CN); Miao WU, Ningbo City (CN); Changgui LIN, Ningbo City (CN); Yingying WANG, Ningbo City (CN); Peiqing ZHANG, Ningbo City (CN); Zhaoxiang QIU, Ningbo City (CN); Ao CUI, Ningbo City (CN); Jinjin CHEN, Ningbo City (CN)

(21) Appl. No.: 18/802,230

(22) Filed: Aug. 13, 2024

(30) Foreign Application Priority Data
Oct. 26, 2023 (CN) 202311394780.8

(51) Int. Cl. G01N 33/38; G01D 21/02; G02F 1/11

(52) U.S. Cl. CPC G01N 33/386; G01D 21/02; G02F 1/11

(57) ABSTRACT

A method for evaluating comprehensive performance of an acousto-optic chalcogenide glass material is provided, which is a comprehensive quantitative evaluation method achieved by an analytic hierarchy process and a radar chart method. Indexes of an evaluation index system of the method include acousto-optic figure of merit, mechanical performance, a thermo-optic coefficient, laser damage resistance, acoustic performance and other important performance. According to the method, the defect of subjective judgment of a decision maker is overcome, and the weights of various performance evaluation indexes of the acousto-optic chalcogenide glass material are more reasonable. Meanwhile, the advantages and disadvantages of the performance of the acousto-optic chalcogenide glass material are evaluated using the radar chart, which intuitively and concisely display whether the performance of the material is balanced or not.

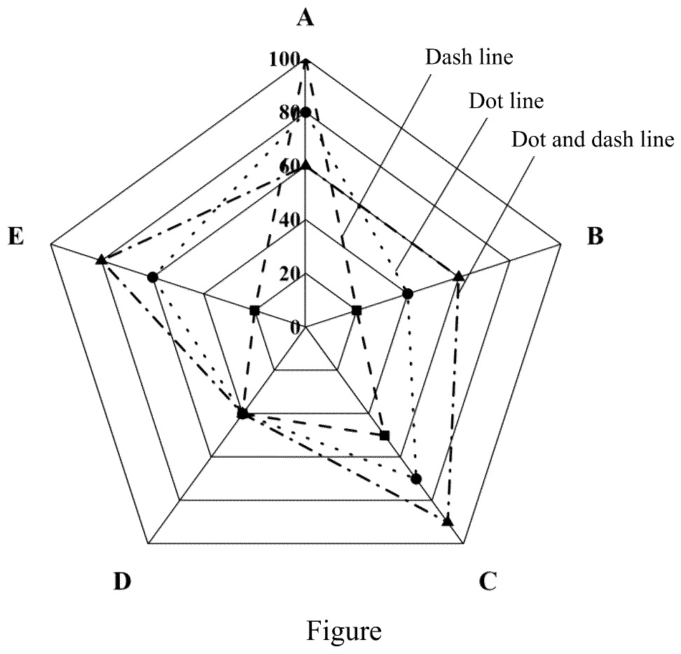

### CROSS-REFERENCE TO RELATED APPLICATION

This patent application claims the benefit and priority of Chinese Patent Application No. 202311394780.8 filed with the China National Intellectual Performance Administration on Oct. 26, 2023, the disclosure of which is incorporated by reference herein in its entirety as part of the present application.

### TECHNICAL FIELD

The present disclosure relates to the technical field of acousto-optic technique, and in particular to a method for evaluating the comprehensive performance of an acousto-optic chalcogenide glass material.

### BACKGROUND

Acousto-optic device which processes the frequency, amplitude and direction of the optical signal using ultrasonic waves has the characteristics of fast transmission and conversion of electrical, acoustic and optical information. Acousto-optic devices represented by acousto-optic modulator, acousto-optic deflector and acousto-optic tunable filter are widely used in the fields of optical information technology, laser technology and radar receiving systems.

Core components of the acousto-optic device mainly include two parts: an ultrasonic transducer (piezoelectric material such as LiNbO₃) and an acousto-optic medium, and the performance improvement of the acousto-optic device depends on acousto-optic medium materials. Acousto-optic crystal materials, such as PbMoO₄, GaAs, TeO₂, etc., have become the main medium materials of the acousto-optic device because of their excellent optical uniformity, high mechanical hardness, good thermal conductivity, low acoustic attenuation coefficient and high laser damage threshold. With the development of industrial technology and national defense technology, the frontier research and development of the acousto-optic device is now developing towards low power consumption, high laser power handling capacity, mid-infrared working band and miniaturization. Traditional acousto-optic crystal materials cannot meet the development needs of high-performance acousto-optic devices due to their low acousto-optic figure of merit (M₂), complex preparation process and difficulty in large-size preparation, so there is an urgent need to develop new acousto-optic medium materials.

Chalcogenide glass materials, due to their advantages of high optical refractive index, extremely high acousto-optic figure of merit M₂, extremely wide infrared transmission range and easy large-scale manufacture, can effectively improve the diffraction efficiency of the acousto-optic device and reduce the power consumption of the device, and thus have great application prospects in the field of acousto-optic devices. Some commercial companies abroad have developed acousto-optic devices based on chalcogenide glass. Compared with acousto-optic crystal materials, common chalcogenide glass, such as As₂S₃ and As₂Se₃, generally has the disadvantages of low mechanical strength, high acoustic attenuation coefficient and low laser damage threshold. In addition, during actual application of the acousto-optic media, the thermal lens effect of the acousto-optic medium materials is also an important index parameter to be considered. However, there is no method for evaluating the comprehensive performance of an acousto-optic chalcogenide glass material in the prior art. Therefore, how to evaluate the comprehensive performance of acousto-optic chalcogenide glass materials with different compositions to meet the practical application of the acousto-optic device has become an urgent problem to be solved.

### SUMMARY

The technical problem to be solved by the present disclosure is as follows: in order to address the gap in the prior art, a method for evaluating the comprehensive performance of an acousto-optic chalcogenide glass material is provided, which provides an important support for the application of the acousto-optic chalcogenide glass material.

The technical solution adopted by the present disclosure for solving the technical problem above is as follows: A method for evaluating the comprehensive performance of an acousto-optic chalcogenide glass material includes the following steps:
S1: determining an evaluation index system for the comprehensive performance of the acousto-optic chalcogenide glass material, where indexes in the evaluation index system comprise the acousto-optic figure of merit, mechanical performance, thermo-optic coefficient, laser damage resistance and acoustic performance, and corresponding index parameters are shown in Table 1:

TABLE 1
A constitution of the evaluation index system for the
comprehensive performance
of the acousto-optic chalcogenide glass material

| Serial number | Index | Index parameter |
|---|---|---|
| 1 | Acousto-optic | Acousto-optic figure |
| | figure of merit | of merit M₂ |
| 2 | Mechanical performance | Vickers hardness H_{V} |
| 3 | | Young's Modulus E |
| 4 | Thermo-optic coefficient | Thermo-optic coefficient dn/dT |
| 5 | Laser damage resistance | Laser damage threshold P |
| 6 | Acoustic performance | Acoustic attenuation |
| | | coefficient α |

S2: grading and scoring the index parameters in the evaluation index system for the comprehensive performance of the acousto-optic chalcogenide glass material, where the scoring rules and performance test methods are shown in Table 2:

TABLE 2
A score chart of the index parameters for the comprehensive
performance of the acousto-optic chalcogenide glass material

| Serial | Index | Test | Score | | | | |
|---|---|---|---|---|---|---|---|
| number | parameter | method | 20 | 40 | 60 | 80 | 100 |
| 1 | Acousto- | BS | M₂ ≤ 100 | 100 < M₂ ≤ | 200 < M₂ ≤ | 400 < M₂ ≤ | M₂ > 600 |
| | optic figure | 7604-1- | | 200 | 400 | 600 | |
| | of merit M₂ | 1992 | | | | | |
| | (×10⁻¹⁸ s³/g) | | | | | | |
| 2 | Vickers | GB/T | Hᵥ ≤ 120 | 120 < Hᵥ ≤ | 150 < Hᵥ ≤ | 180 < Hᵥ ≤ | Hᵥ > 210 |
| | hardness Hᵥ | 5266- | | 150 | 180 | 210 | |
| | (kg/mm²) | 2006 | | | | | |
| 3 | Young's | GB/T | E ≤ 15 | 15 < E ≤ 17 | 17 < E ≤ 19 | 19 < E ≤ 25 | E > 25 |
| | Modulus E | 38897- | | | | | |
| | (GPa) | 2020 | | | | | |
| 4 | Thermo- | GB/T | dn/dT > | 40 < dn/dT ≤ | 20 < dn/dT ≤ | 10 < dn/dT ≤ | dn/dT ≤ |
| | optic | 42657- | 80 | 80 | 40 | 20 | 10 |
| | coefficient | 2023 | | | | | |
| | dn/dT | | | | | | |
| | (×10⁻⁶/K) | | | | | | |
| 5 | Laser | GB/T | P ≤ 2 | 2 < P < 4.5 | 4.5 < P ≤ 6 | 6 < P ≤ 10 | P > 10 |
| | damage | 16601.2- | | | | | |
| | threshold | 2017 | | | | | |
| | P(W/mm²) | | | | | | |
| 6 | Acoustic | GB/T | a > 10 | 7 < a ≤ 10 | 4 < a ≤ 7 | 2 < a ≤ 4 | a ≤ 2 |
| | attenuation | 5266- | | | | | |
| | coefficient a | 2006 | | | | | |
| | (cm/dB@, | | | | | | |
| | 100 MHz) | | | | | | |

S3: using an analytic hierarchy process (i.e., AHP) to obtain weights of the index parameters of the acousto-optic figure of merit, mechanical performance, thermo-optic coefficient, laser damage resistance and acoustic performance;
S4: calculating a grading evaluation result of the comprehensive performance of the acousto-optic chalcogenide glass material: weighting a score of the acousto-optic figure of merit, a score of the mechanical performance, a score of the thermo-optic coefficient, a score of the laser damage resistance, and a score of the acoustic performance according to the weights to obtain an overall score of the comprehensive performance of the acousto-optic chalcogenide glass material, thereby evaluating whether the comprehensive performance of the acousto-optic chalcogenide glass material is qualified or not;
S5: drawing a radar chart according to grading score results of respective index parameters in Step S2; and
S6: evaluating the performance of the acousto-optic chalcogenide glass material according to the radar chart.

The indexes in the evaluation index system of the evaluation method provided by the present disclosure include acousto-optic figure of merit, mechanical performance, thermo-optic coefficient, laser damage resistance and acoustic performance, and the specific corresponding index parameters are the acousto-optic figure of merit, Vickers hardness and Young's modulus, thermo-optic coefficient, laser damage threshold and acoustic attenuation coefficient. The acousto-optic figure of merit mainly reflects the diffraction efficiency of acousto-optic material, and a testing method for the acousto-optic figure of merit is BS 76Apr. 1, 1992. Vickers hardness and Young's modulus mainly reflect the machinability of the acousto-optic material, i.e., the deformation resistance, and there is little difference in importance, so the Vickers hardness and Young's modulus are treated equally in the evaluation. A testing method for the Vickers hardness is GB/T 5266-2006 and a testing method for the Young's modulus is GB/T 38897-2020. The thermo-optic coefficient reflects thermal stability of the acousto-optic material under laser irradiation for a long time (thermal lens effect). Generally, the thermo-optic coefficient of the acousto-optic chalcogenide glass material is between 0-100×10⁻⁶/K, and a testing method for the thermo-optic coefficient is generally GB/T 42657-2023. The laser damage threshold of the laser damage resistance of the acousto-optic chalcogenide glass materials is the maximum laser energy density or laser intensity that the acousto-optic material can withstand before the damage occurs, which determines whether the acousto-optic device can work for a long time under high-power laser. The testing method for the laser damage threshold is GB/T 16601.2-2017. The acoustic attenuation coefficient refers to the absorption of sound waves by the acousto-optic material, which is inversely proportional to the square of frequency, and a testing method for the acoustic attenuation coefficient is GB/T 5266-2006.

In some embodiments, the process of Step S3 is as follows:
sorting evaluation indexes of the comprehensive performance of the acousto-optic chalcogenide glass material in terms of importance to construct a decision matrix A;

$$A = [ \begin{matrix}{a}_{11} & … & {a}_{1 j} & … & {a}_{1 n} \\ ⋮ & & ⋮ & & ⋮ \\ {a}_{i 1} & … & {a}_{ij} & … & {a}_{in} \\ ⋮ & & ⋮ & & ⋮ \\ {a}_{n 1} & … & {a}_{nj} & … & {a}_{nn}\end{matrix} ] , {a}_{ii} = 1 , {a}_{ij} = 1 / {a}_{ji} , {a}_{ji} \neq 0 ,$$

where aᵢⱼ indicates the importance of an i-th performance evaluation index compared with a j-th performance evaluation index, i and j are positive integers, i=1, 2, . . . n, and j=1, 2, . . . n.

A weight of each evaluation index of the comprehensive performance of the acousto-optic chalcogenide glass material is obtained by formula (1):

$$( A - {λ}_{max} I ) ω = 0 , \qquad ( 1 )$$

where λₘₐₓ is a maximum eigenvalue of the matrix A, I is a unit vector, Ω is an eigenvector of the matrix A, which is the weights of the evaluation indexes of the comprehensive performance of the acousto-optic chalcogenide glass material.

Further, in Step S3, after obtaining the weights of the evaluation indexes of the comprehensive performance of the acousto-optic chalcogenide glass material, formula (2) is used for weight check:

$$CI = ( {λ}_{max} - n ) / ( n - 1 ) = 0 , \qquad ( 2 )$$

where CI is a consistency index, when CI is less than 0.1, the obtained weight is considered reasonable.

In some embodiments, in Step S4, an evaluation criterion of the comprehensive performance of the acousto-optic chalcogenide glass material is as follows: a total score of greater than or equal to 80 indicates that the comprehensive performance of the acousto-optic chalcogenide glass material is excellent; a total score of greater than or equal to 60 and less than 80 indicates that the comprehensive performance of the acousto-optic chalcogenide glass material is qualified; and a total score of less than 60 indicates that the comprehensive performance of the acousto-optic chalcogenide glass material does not meet the requirements.

Compared with the prior art, the present disclosure has the following advantages: a method for evaluating the comprehensive performance of an acousto-optic chalcogenide glass material, which is proposed for the first time, is comprehensive quantitative evaluation method achieved by an analytic hierarchy process and a radar chart method. Indexes of an evaluation index system of the method include acousto-optic figure of merit, mechanical performance, thermo-optic coefficient, laser damage resistance, acoustic performance and other important performance. According to the method, weights of the performance of the acousto-optic chalcogenide glass material are calculated using the analytic hierarchy process, so the defect of subjective judgment of a decision maker is overcome, and the weights of various performance evaluation indexes of the acousto-optic chalcogenide glass material are more reasonable. Meanwhile, the advantages and disadvantages of the performance of the acousto-optic chalcogenide glass material are evaluated using the radar chart, which intuitively and concisely display whether the performance of the material is balanced or not. The method for evaluating the comprehensive performance of an acousto-optic chalcogenide glass material provided by the present disclosure provides an important support for the application of acousto-optic chalcogenide glass materials.

### BRIEF DESCRIPTION OF THE DRAWINGS

Figure is a radar chart obtained by evaluating the comprehensive performance of three acousto-optic chalcogenide glass materials according to an embodiment.

### DETAILED DESCRIPTION OF THE EMBODIMENTS

To better describe the present disclosure and facilitate the understanding of the technical solution of the present disclosure conveniently, the following detailed description of the present disclosure is provided with reference to specific embodiments.

Taking three acousto-optic chalcogenide glass materials, i.e., As₂Se₃, Ge₂₀Sb₁₅Se₆₅ and Ge₃₃As₁₂Se₅₅, as examples, the comprehensive performance is evaluated by a method provided by the present disclosure, with specific steps 1 to 5 as follows.

In step 1, acousto-optic figure of merit, Vickers hardness, Young's modulus, thermo-optic coefficients, laser-induced damage thresholds and acoustic attenuation coefficients of the three acousto-optic chalcogenide glass materials, i.e., As₂Se₃, Ge₂₀Sb₁₅Se₆₅ and Ge₃₃As₁₂Se₅₅, are selected as index parameters for evaluation, and measured values of the index parameters are obtained by measuring according to the testing methods listed in Table 1.

In step 2, according to the scoring rules in Table 2, the comprehensive performance of the three acousto-optic chalcogenide glass materials is graded based on the measured values of their index parameters.

The measured values and grading-score results of the index parameters of the three acousto-optic chalcogenide glass materials are as shown in Table 3.

TABLE 3
A score chart of index parameters of the comprehensive
performance of three acousto-optic chalcogenide glass materials

| | | Aₛ₂Se₃ | Ge₂₀Sb₁₅Se₆₅ | Ge₃₃As₁₂Se₅₅ |
|---|---|---|---|---|
| Index parameter | | glass | glass | glass |
| Acousto-optic figure | Measured value | 676.9 | 355.5 | 231.3 |
| of merit M₂ | Score | 100 | 80 | 60 |
| (×10⁻¹⁸ s³/g) | | | | |
| Vickers hardness H_{V} | Measured value | 135 | 165 | 223 |
| (kg/mm²) | Score | 40 | 60 | 100 |
| Young's Modulus E | Measured value | 18.52 | 19.72 | 19.51 |
| (GPa) | Score | 60 | 80 | 80 |
| Thermo-optic | Measured value | 49.8 | 54.2 | 69.1 |
| coefficient dn/dT | Score | 40 | 40 | 40 |
| (×10⁻⁶/K) | | | | |
| Laser damage | Measured value | 2.51 | 4.24 | 5.21 |
| threshold P | Score | 20 | 40 | 60 |
| (W/mm²) | | | | |
| Acoustic attenuation | Measured value | 8.79 | 5.95 | 3.98 |
| coefficient | Score | 20 | 60 | 80 |
| α(cm/ | | | | |
| dB@100 MHz) | | | | |

In step 3, an analytic hierarchy process (i.e., AHP) is used to obtain weights of the index parameters of the acousto-optic figure of merit, mechanical performance, thermo-optic coefficient, laser damage resistance and acoustic performance, thereby constructing an importance sorting table, as shown in Table 4. When analyzing the weight of each index, it is preferred to assign the importance according to comparative scales of 1-9. Scale 1 indicates that two performance indexes are equally important; Scale 3 indicating that the former is slightly more important than the latter upon comparison therebetween, Scale 5 indicates that the former is obviously more important than the latter upon comparison therebetween; Scale 7 indicates that the former is intensively more important than the latter upon comparison therebetween; and Scale 9 indicates that the former is extremely more important than the latter upon comparison therebetween. Scales 2, 4, 6 and 8 represent the intermediate values of the above adjacent judgment results. If a ratio of the importance of a performance index i to a performance index j is a aᵢⱼ, the ratio of the importance of the performance index j to the performance index i is aᵢⱼ=1/aᵢⱼ.

The weights of performance evaluation indexes obtained by formula (1) can be normalized to obtain ω=[0.21, 0.32, 0.14, 0.25, 0.08], which means that the indexes of the acousto-optic figure of merit, laser damage resistance, mechanical performance, thermo-optic coefficient and acoustic performance respectively account for 21%, 32%, 14%, 25% and 8% in the comprehensive performance evaluation index system. In order to further determine the rationality of the weights of evaluation indexes, formula (2) is used for weight check to obtain CI=0.041<0.1, indicating that the calculated weight is reasonable.

TABLE 4
An importance sorting of performance indexes of acousto-optic chalcogenide
glass materials

| | Acousto-optic | Laser damage | Mechanical | Thermo-optic | Acoustic |
|---|---|---|---|---|---|
| Index | figure of merit | resistance | performance | coefficient | performance |
| Acousto-optic | 1 | ⅓ | 2 | 1 | 3 |
| figure of merit | | | | | |
| Laser damage | 3 | 1 | 2 | 1 | 3 |
| resistance | | | | | |
| Mechanical | ½ | ½ | 1 | ½ | 2 |
| performance | | | | | |
| Thermo-optic | 1 | 1 | 2 | 1 | 3 |
| coefficient | | | | | |
| Acoustic | ⅓ | ⅓ | ½ | ⅓ | 1 |
| performance | | | | | |

In step 4, a score of the acousto-optic figure of merit, a score of the mechanical performance, a score of the thermo-optic coefficient, a score of the laser damage resistance and a score of the acoustic performance in Table 1 are weighted according to the index weights above, where the score of the mechanical performance=(a score of Vickers hardness+a score of Young's modulus)/2, and the total score=the score of acousto-optic figure of merit×21%+the score of mechanical performance×14%+the score of thermo-optic coefficient×25%+the score of laser damage resistance×32%+score of the acoustic performance×8%.

For the As₂Se₃ glass the comprehensive performance grading evaluation result is 44.7 points. For Ge₂₀Sb₁₅Se₆₅ glass the comprehensive performance grading evaluation result is 54.2 points. For Ge₃₃As₁₂Se₅₅ glass the comprehensive performance grading evaluation result is 60.8 points. It can be seen that Ge₃₃As₁₂Se₅₅ glass has the best comprehensive acousto-optic performance which is qualified.

In step 5, a radar chart is drawn. The corresponding points of various index scores in Table 1 are marked on the index axis to obtain five points A, B, C, D and E in turn, which respectively represent the acousto-optic figure of merit index, laser damage resistance index, mechanical performance index, thermo-optic coefficient index and acoustic performance index. The five points are connected to get a polygonal radar chart, and different acousto-optic chalcogenide glass materials are represented using lines with different line-types. The drawn radar chart is shown in Figure. The acousto-optic chalcogenide glass materials corresponding to a dash line, a dot line and a dot-dash line in Figure are As₂Se₃, Ge₂₀Sb₁₅Se₆₅, Ge₃₃As₁₂Se₅₅ glasses, respectively.

It can be clearly seen from Figure that the performance index of the As₂Se₃ acousto-optic chalcogenide glass material is uneven, especially the performance indexes of the laser damage resistance and thermo-optic coefficient are low, which makes the radar chart deviate from a regular pentagon shape. The Ge₃₃As₁₂Se₅₅ glass has the most balanced performance indexes. On the whole, the thermo-optic coefficients of the three acousto-optic chalcogenide glass materials are poor.

According to the comprehensive performance evaluation criterion of the acousto-optic chalcogenide glass materials, the comprehensive performance of As₂Se₃, Ge₂₀Sb₁₅Se₆₅ and Ge₃₃As₁₂Se₅₅ glasses, is evaluated. For the As₂Se₃ and Ge₂₀Sb₁₅Se₆₅ glasses, the thermo-optic coefficient, acoustic performance and laser damage resistance of the Ge₂₀Sb₁₅Se₆₅ glasses are slightly poor, while the acousto-optic figure of merit M₂ of the As₂Se₃ glass is excellent, but other performance indexes of the As₂Se₃ glass are unqualified. Therefore, the requirements of the acousto-optic devices for the acousto-optic medium material cannot be satisfied by As₂Se₃ and Ge₂₀Sb₁₅Se₆₅ glasses. For the Ge₃₃As₁₂Se₅₅ glass, all the indexes are developed in a balanced way, and the comprehensive score is 60.8, so the comprehensive performance of the Ge₃₃As₁₂Se₅₅ glass is qualified, and the Ge₃₃As₁₂Se₅₅ has been used as a medium material in commercial acousto-optic devices.

In order to verify the rationality of the method for evaluating the comprehensive performance of the acousto-optic chalcogenide glass material, As₂Se₃, Ge₂₀Sb₁₅Se₆₅ and Ge₃₃As₁₂Se₅₅ glasses, are made into acousto-optic modulators to verify the device performance. The three acousto-optic modulators have the completely same design process parameters except for the different acousto-optic medium materials. The measured data are shown in Table 5. As the tolerant optical power of the acousto-optic modulator made of As₂Se₃ glass is too low, the acousto-optic modulator cannot work for a long time and cannot be applied in practice. The acousto-optic modulators made of any one of the two acousto-optic chalcogenide glass materials, Ge₂₀Sb₁₅Se₆₅ and Ge₃₃As₁₂Se₅₅, have high extinction ratio. The acousto-optic modulator made of the Ge₃₃As₁₂Se₅₅ chalcogenide glass, except for slightly lower diffraction efficiency, has higher tolerant optical power, lower insertion loss and shorter light pulse rise time, and has been used as a commercial acousto-optic modulator at present. The verification results are consistent with evaluation results obtained according to the evaluation method provided by the present disclosure.

TABLE 5
A performance test table of chalcogenide glass acousto-optic modulators

| Acousto- | Tolerant | Insertion | Extinction | Diffraction | Optical |
|---|---|---|---|---|---|
| optic | optical power | loss | ratio | efficiency | pulse |
| material | (W/mm²) | (dB) | (dB) | (%) | rise-time |
| Ge₃₃As₁₂Se₅₅ | ≥4.26 | 1.38 | 67 | 68.1 | 48 |
| glass | | | | | |
| Ge₂₀Sb₁₅Se₆₅ | ≤4.24 | 2.4 | 68 | 73.4 | 56 |
| glass | | | | | |
| As₂Se₃ glass | ≤2.51 | 2.2 | 66 | 76.9 | 57 |

What is claimed is:

1. A method for evaluating comprehensive performance of an acousto-optic chalcogenide glass material, comprising following steps:
S1: determining an evaluation index system for the comprehensive performance of the acousto-optic chalcogenide glass material, wherein indexes in the evaluation index system comprise an acousto-optic figure of merit, a mechanical performance, a thermo-optic coefficient, a laser damage resistance and an acoustic performance, and corresponding index parameters are shown in Table 1:
TABLE 1
constitution of the evaluation index system for
the comprehensive performance of the
acousto-optic chalcogenide glass material

| Serial number | Index | Index parameter |
|---|---|---|
| 1 | Acousto-optic | Acousto-optic figure |
| | figure of merit | of merit M₂ |
| 2 | Mechanical | Vickers hardness H_{V} |
| 3 | performance | Young's Modulus E |
| 4 | Thermo-optic coefficient | Thermo-optic coefficient dn/dT |
| 5 | Laser damage resistance | Laser damage threshold P |
| 6 | Acoustic performance | Acoustic attenuation |
| | | coefficient α |

S2: grading and scoring the index parameters in the evaluation index system for the comprehensive performance of the acousto-optic chalcogenide glass material, wherein scoring rules and performance test methods are shown in Table 2:
TABLE 2
A score chart of the index parameters for the comprehensive
performance of the acousto-optic chalcogenide glass material

| Serial | Index | Test | Score | | | | |
|---|---|---|---|---|---|---|---|
| number | parameter | method | 20 | 40 | 60 | 80 | 100 |
| 1 | Acousto- | BS | M₂ ≤ 100 | 100 < M₂ ≤ | 200 < M₂ ≤ | 400 < M₂ ≤ | M₂ > 600 |
| | optic figure | 7604-1- | | 200 | 400 | 600 | |
| | of merit M₂ | 1992 | | | | | |
| | (×10⁻¹⁸ s³/g) | | | | | | |
| 2 | Vickers | GB/T | Hᵥ ≤ 120 | 120 < Hᵥ ≤ | 150 < Hᵥ ≤ | 180 < Hᵥ ≤ | Hᵥ > 210 |
| | hardness Hᵥ | 5266- | | 150 | 180 | 210 | |
| | (kg/mm²) | 2006 | | | | | |
| 3 | Young's | GB/T | E ≤ 15 | 15 < E ≤ 17 | 17 < E ≤ 19 | 19 < E ≤ 25 | E > 25 |
| | Modulus E | 38897- | | | | | |
| | (GPa) | 2020 | | | | | |
| 4 | Thermo- | GB/T | dn/dT > | 40 < dn/dT ≤ | 20 < dn/dT ≤ | 10 < dn/dT ≤ | dn/dT ≤ |
| | optic | 42657- | 80 | 80 | 40 | 20 | 10 |
| | coefficient | 2023 | | | | | |
| | dn/dT | | | | | | |
| | (×10⁻⁶/K) | | | | | | |
| 5 | Laser | GB/T | P ≤ 2 | 2 < P ≤ 4.5 | 4.5 < P ≤ 6 | 6 < P ≤ 10 | P > 10 |
| | damage | 16601.2- | | | | | |
| | threshold | 2017 | | | | | |
| | P(W/mm²) | | | | | | |
| 6 | Acoustic | GB/T | a > 10 | 7 < a ≤ 10 | 4 < a ≤ 7 | 2 < a ≤ 4 | a ≤ 2 |
| | attenuation | 5266- | | | | | |
| | coefficient a | 2006 | | | | | |
| | (cm/dB@, | | | | | | |
| | 100 MHz) | | | | | | |

S3: using an analytic hierarchy process (i.e., AHP) to obtain weights of the index parameters of the acousto-optic figure of merit, the mechanical performance, the thermo-optic coefficient, the laser damage resistance and the acoustic performance;
S4: calculating a grading evaluation result of the comprehensive performance of the acousto-optic chalcogenide glass material: weighting a score of the acousto-optic figure of merit, a score of the mechanical performance, a score of the thermo-optic coefficient, a score of the laser damage resistance, and a score of the acoustic performance according to the weights to obtain an overall score of the comprehensive performance of the acousto-optic chalcogenide glass material, thereby evaluating whether the comprehensive performance of the acousto-optic chalcogenide glass material is qualified or not;
S5: drawing a radar chart according to grading score results of respective index parameters in Step S2; and
S6: evaluating of the performance of the acousto-optic chalcogenide glass material according to the radar chart.

2. The method according to claim 1, wherein Step S3 comprises following steps:
sorting evaluation indexes of the comprehensive performance of the acousto-optic chalcogenide glass material in terms of importance to construct a decision matrix A;
$$A = [ \begin{matrix}{a}_{11} & … & {a}_{1 j} & … & {a}_{1 n} \\ ⋮ & & ⋮ & & ⋮ \\ {a}_{i 1} & … & {a}_{ij} & … & {a}_{in} \\ ⋮ & & ⋮ & & ⋮ \\ {a}_{n 1} & … & {a}_{nj} & … & {a}_{nn}\end{matrix} ] , {a}_{ii} = 1 , {a}_{ij} = 1 / {a}_{ji} , {a}_{ji} \neq 0$$
wherein aᵢⱼ indicates importance of an i-th performance evaluation index compared with a j-th performance evaluation index, i and j are positive integers, i=1, 2, . . . n, and j=1, 2, . . . n;
a weight of each evaluation index of the comprehensive performance of the acousto-optic chalcogenide glass material is obtained by formula (1):
$$( A - {λ}_{max} I ) ω = 0 \qquad ( 1 )$$
wherein λₘₐₓ is a maximum eigenvalue of the matrix A, I is a unit vector, ω is an eigenvector of the matrix A, which is weights of the evaluation indexes of the comprehensive performance of the acousto-optic chalcogenide glass material.

3. The method according to claim 2, wherein in Step S3, after obtaining the weights of the evaluation indexes of the comprehensive performance of the acousto-optic chalcogenide glass material, formula (2) is used for weight check:
$$CI = ( {λ}_{max} - n ) / ( n - 1 ) = 0 \qquad ( 2 )$$
wherein CI is a consistency index, when CI is less than 0.1, the obtained weight is considered reasonable.

4. The method according to claim 1, wherein in Step S4, an evaluation criterion of the comprehensive performance of the acousto-optic chalcogenide glass material is as follows: a total score of greater than or equal to 80 indicates that the comprehensive performance of the acousto-optic chalcogenide glass material is excellent; a total score of greater than or equal to 60 and less than 80 indicates that the comprehensive performance of the acousto-optic chalcogenide glass material is qualified; and a total score of less than 60 indicates that the comprehensive performance of the acousto-optic chalcogenide glass material does not meet requirements.

5. The method according to claim 4, wherein Step S3 comprises following steps:
sorting evaluation indexes of the comprehensive performance of the acousto-optic chalcogenide glass material in terms of importance to construct a decision matrix A;
$$A = [ \begin{matrix}{a}_{11} & … & {a}_{1 j} & … & {a}_{1 n} \\ ⋮ & & ⋮ & & ⋮ \\ {a}_{i 1} & … & {a}_{ij} & … & {a}_{in} \\ ⋮ & & ⋮ & & ⋮ \\ {a}_{n 1} & … & {a}_{nj} & … & {a}_{nn}\end{matrix} ] , {a}_{ii} = 1 , {a}_{ij} = 1 / {a}_{ji} , {a}_{ji} \neq 0$$
wherein aᵢⱼ indicates importance of an i-th performance evaluation index compared with a j-th performance evaluation index, i and j are positive integers, i=1, 2, . . . n, and j=1, 2, . . . n;
a weight of each evaluation index of the comprehensive performance of the acousto-optic chalcogenide glass material is obtained by formula (1):
$$( A - {λ}_{max} I ) ω = 0 \qquad ( 1 )$$
wherein λₘₐₓ is a maximum eigenvalue of the matrix A, I is a unit vector, ω is an eigenvector of the matrix A, which is weights of the evaluation indexes of the comprehensive performance of the acousto-optic chalcogenide glass material.

6. The method according to claim 5, wherein in Step S3, after obtaining the weights of the evaluation indexes of the comprehensive performance of the acousto-optic chalcogenide glass material, formula (2) is used for weight check:
$$CI = ( {λ}_{max} - n ) / ( n - 1 ) = 0 \qquad ( 2 )$$
wherein CI is a consistency index, when CI is less than 0.1, the obtained weight is considered reasonable.
